(19) United States
(12) Patent Application Publication
(10) Pub. No.: US 2010/0317032 A1
(43) Pub. Date: Dec. 16, 2010

(54) METHOD FOR DETECTING ANTIGEN AND ANTIGEN DETECTION DEVICE

(75) Inventors: Koichi MINAMI, Kanagawa (JP); Hirohiko TSUZUKI, Kanagawa (JP); Hiroshi UEDA, Tokyo (JP); Masaki IHARA, Tokyo (JP)

Correspondence Address: Solaris Intellectual Property Group, PLLC, 401 Holland Lane, Suite 407, Alexandria, VA, 22314, US

(73) Assignees: FUJIFILM CORPORATION, TOKYO (JP); THE UNIVERSITY OF TOKYO, TOKYO (JP)

(21) Appl. No.: 12/796,676

(22) Filed: Jun. 9, 2010

(30) Foreign Application Priority Data
Jun. 10, 2009 (JP) 2009-139119
Oct. 8, 2009 (JP) 2009-234582

(51) Int. Cl. G01N 33/53

(52) U.S. Cl. 435/7.9

(57) ABSTRACT

Provided is a method for detecting an antigen in a sample, the method including: bringing an unlabeled polypeptide and a labeled polypeptide into contact with an antigen in a sample, the unlabeled polypeptide being one of a pair including a VH-region polypeptide and a VL-region polypeptide which are separate and capable of cooperatively recognizing the antigen, and the labeled polypeptide being the other of the pair including the separate VH-region polypeptide and the VL-region polypeptide and being labeled with an environmentally-responsive substance at a site where the environmentally-responsive substance does not inhibit binding of the antigen, and detecting a change in the environmentally-responsive substance caused by a change in the environment around the labeled polypeptide after the contact. Also provided is an antibody fragment polypeptide set including the unlabeled polypeptide and the labeled polypeptide.

### CROSS-REFERENCE TO RELATED APPLICATION

This application claims priority under 35 USC 119 from Japanese Patent Application Nos. 2009-139119 filed on Jun. 10, 2009 and 2009-234582 filed on Oct. 8, 2009, the disclosures of which are incorporated by reference herein.

### BACKGROUND OF THE INVENTION

1. Field of the Invention

The present invention relates to a method for detecting an antigen and an antigen detection device.

2. Description of the Related Art

As systems for detecting various proteins as target substances with high accuracy, immunoassays such as ELISA are well known. In these immunoassays, proteins are used as target substances and antibodies specific to these proteins are used as detection substances, and the target substances are detected with high sensitivity based on specific interactions between the target substances and the detection substances. Although various improvements have been made to ELISA in view of sensitivity and operability, there is a growing demand, in recent years, for simple detection of a large amount of samples in a short time with high sensitivity.

As a system using specific binding due to antigen-antibody reaction as in the above technology, a reagent for an immunoassay using fluorescence resonance energy transfer (FRET) between two types of fluorescence-labeled antibody fragments is disclosed in Japanese Patent Application Laid-Open (JP-A) No. 10-78436. When using this reagent for an immunoassay, if the antigen does not exist, the antigen-antibody complex is not formed and FRET does not occur, and if the antigen exists, the antigen-antibody complex is formed and FRET occurs. As a result, the presence or absence of the antigen can be rapidly and simply detected.

However, in this method, it is necessary to label each of the two types of antibody fragments with a fluorescent dye. Further, in cases where detection of an antigen is carried out by FRET, it is necessary to carry out the two kinds of fluorescent labeling in the optimum positional relationship at which FRET is likely to occur, making molecular design difficult. Further, since luminescence from each of the two kinds of fluorescent dyes when not bound to the antibody contributes to background luminescence or background noise, the ratios of concentration of the two labeling dyes must be maintained optimally in consideration of the concentration of the antigen contained in the measurement sample. Thus, in a detection system using two types of fluorescence-labeled antibody fragments, the individual labeling and adjustment of their ratios of concentration are laborious, and without adjusting these ratios, sensitivity of detection of the antigen may decrease.

On the other hand, various fluorescent probes for monitoring with high sensitivity over a long time have been developed.

For example, in the Abstract of the 57th Annual Meeting of the Japan Society for Analytical Chemistry, p. 97, E3017, 2008, is disclosed a fluorescent molecular probe for detection of a vascular endothelial cell growth factor (VEGF) for monitoring VEGF simply and rapidly over a long time. This fluorescent molecular probe for VEGF is constituted by a peptide chain as a VEGF binding site in a VEGF receptor and a fluorescent chromophore. It is described that, when this fluorescent molecular probe and VEGF are mixed together at room temperature in a buffer, the environment near the fluorescent chromophore becomes hydrophobic and the fluorescence intensity increases.

However, since the fluorescent molecular probe uses a peptide chain as the binding site in the VEGF receptor, a site near the binding site for VEGF is labeled with the fluorescent chromophore. Labeling at such a site may affect the binding capacity of the fluorescent molecular probe to VEGF. Further, since designing such a peptide is difficult, each peptide needs to be individually designed and produced, which leads to increased costs. Further, since production thereof is laborious, its versatility is greatly limited.

### SUMMARY OF THE INVENTION

Thus, neither the technique of labeling by two kinds of fluorescent dyes nor that of designing of individual molecular probes, allows versatile, accurate and inexpensive measurement of various subject compounds based on an antigen-antibody reaction.

The present invention was made under such circumstances and aims to provide an antigen detection method capable of detecting various subject compounds accurately and inexpensively with high versatility, and an antibody-fragment-polypeptide set used therewith.

According to a first aspect of the invention, a method for detecting an antigen in a sample includes:

bringing an unlabeled polypeptide and a labeled polypeptide into contact with an antigen in a sample, the unlabeled polypeptide being one of a pair including a VH-region polypeptide and a VL-region polypeptide which are separate and capable of cooperatively recognizing the antigen, and the labeled polypeptide being the other of the pair including the separate VH-region polypeptide and VL-region polypeptide and being labeled with an environmentally-responsive substance at a site where the environmentally-responsive substance does not inhibit binding of the antigen, and

detecting a change in the environmentally-responsive substance caused by a change in the environment around the labeled polypeptide after the contact.

According to a second aspect of the invention, an antibody fragment polypeptide set includes:

an unlabeled polypeptide which is one of a pair including a VH-region polypeptide and a VL-region polypeptide which are separate and capable of cooperatively recognizing an antigen, and

a labeled polypeptide which is the other of the pair including the VH-region polypeptide and the VL-region polypeptide and which is labeled with an environmentally-responsive substance at a site where the environmentally-responsive substance does not inhibit binding of the antigen.

According to a third aspect of the invention, a kit for detecting an antigen includes the antibody fragment polypeptide set according to the second aspect.

According to a fourth aspect of the invention, an antigen detection device includes:

the antibody fragment polypeptide set according to the second aspect, and

a detection unit that, in a case in which a complex is formed by contact of the unlabeled polypeptide and the labeled polypeptide with the antigen, detects a change in the environmentally-responsive substance caused by a change in the environment around the labeled polypeptide after the contact.

According to a fifth aspect of the invention, an immobilization support includes:

a support; and

the antibody fragment polypeptide set according to the second aspect,

wherein the unlabeled polypeptide and the labeled polypeptide are independently immobilized on the support in a positional relationship which allows binding of the unlabeled polypeptide and the labeled polypeptide to the antigen.

### BRIEF DESCRIPTION OF THE DRAWINGS

FIG. 1A is a conceptual diagram showing an example of the antigen detection device of the present invention;

FIG. 1B is a conceptual diagram showing a complex to be detected by the antigen detection device of the present invention;

FIG. 2 is a conceptual diagram showing another example of the antigen detection device of the present invention; and

FIG. 3 is a preparation scheme of an expression vector used in the Examples section of the specification of the present invention.

### DETAILED DESCRIPTION OF THE INVENTION

According to an exemplary embodiment of the invention, the method for detecting an antigen in a sample includes:

bringing an unlabeled polypeptide and a labeled polypeptide into contact with an antigen in a sample, the unlabeled polypeptide being one of a pair of separate VH-region polypeptide and VL-region polypeptide capable of cooperatively recognizing the antigen, and the labeled polypeptide being the other of the pair of the separate VH-region polypeptide and VL-region polypeptide and being labeled with an environmentally-responsive substance at a site where the environmentally-responsive substance does not inhibit binding of the antigen (hereinafter referred to as “contact step”); and

detecting a change in the environmentally-responsive substance caused by a change in the environment around the labeled polypeptide after the contact (hereinafter referred to as “detection step”).

In the antigen detection method of the present invention, one of a pair of separate VH-region and VL-region polypeptides capable of cooperatively recognizing one type of antigen is used as the labeled polypeptide labeled with an environmentally-responsive substance at a site where the environmentally-responsive substance does not inhibit binding of the antigen, and the other one of the pair of separate VH-region and VL-region polypeptides is used as the unlabeled polypeptide. The VH-region polypeptide and the VL-region polypeptide exist independently from each other, and they only cooperatively recognize an antigen in cases where the antigen exists, thereby coming closer to each other. In the present invention, a change occurs in the environmentally-responsive substance due to a change in the environment around the labeled polypeptide caused when three molecules, that is, the antigen, labeled polypeptide and unlabeled polypeptide are closely located to each other; and detection of the antigen can be performed based on the degree of the change in the environmentally-responsive substance. As a result, the present invention enables detection of various antigen species such as a low molecular weight compound or a protein.

Further, in the invention, since only one of the VH-region polypeptide and VL-region polypeptide which form an antibody molecule as a pair is required to be labeled with an environmentally-responsive substance, the influence of labeling on the antigen affinity can be reduced, and the cost and labor of production can be reduced.

Herein, the “separate VH-region polypeptide and VL-region polypeptide” means that the VH-region polypeptide and the VL-region polypeptide are not linked to each other (for example, by a disulfide bond).

The present invention is described below.

In the contact step in the method of the present invention for detecting an antigen, an unlabeled polypeptide and a labeled polypeptide are brought into contact with an antigen in a sample, the unlabeled polypeptide being one of a pair of separate VH-region and VL-region polypeptides which are capable of cooperatively recognizing one type of antigen, and the labeled polypeptide being the other of the pair of VH-region and VL-region polypeptides and being labeled with an environmentally-responsive substance at a site where the environmentally-responsive substance does not inhibit binding of the antigen.

(1) VH-Region Polypeptide and VL-Region Polypeptide

The length of VH-region polypeptide and VL-region polypeptide may be either longer or shorter than the VH region and VL region of the antibody, respectively, as long as they can bind to a target antigen in association with each other. These polypeptides can be produced from a monoclonal antibody made by a hybridoma technique by using a conventional method. For example, the polypeptides can be obtained as follows.

First, a monoclonal antibody capable of recognizing a desired target substance is produced by a known method. The gene encoding the variable region of this antibody is then specified by a method using a cDNA library and hybridization technique, followed by cloning this gene into a vector. The sequence encoding the VH and/or VL region is then obtained from this recombinant vector, and this sequence fragment is subcloned into an expression vector. By expressing this gene in host cells, the required amount of VH- and/or VL-region polypeptide(s) can be obtained.

In order to obtain the VH/VL coding sequence from the antibody gene, the desired sequence region may be isolated by cleavage with a restriction enzyme and then amplified in a cloning vector, or the desired sequence may be amplified by PCR. When VH and/or VL are/is expressed in host cells, a gene encoding any reporter molecule may also be cloned into the expression vector and VH and/or VL can be expressed as a fusion protein or a chimeric protein with the reporter molecule.

In addition to the above methods, VH and/or VL can be obtained by proteolysis of the antibody molecule using a protease. This method has an advantage of saving time and effort on the gene cloning.

The VH-region polypeptide and the VL-region polypeptide may each be a fusion product with a biomolecule. Such a fusion product has an advantage of improving the stability.

The biomolecule which can be fused with the VH-region polypeptide or the VL-region polypeptide is not particularly restricted, and examples thereof include alkaline phosphatase, protein G, eGFP, eYFP, β-galactosidase, GST, chitin binding protein (CBP), NusA, thioredoxin, DsbA, DsbC, and maltose binding protein (MBP). Among these, in order to further increase stability, MBP is preferably used.

These fusion products can be produced by a conventional method. For example, the fusion product can be obtained by incorporating the gene encoding the biomolecule into a vector at the time of the above-described gene cloning so as to be expressed simultaneously, or by adding a linker to the VH-region polypeptide or the VL-region polypeptide to fuse with the biomolecule. The method for producing the fusion product can be appropriately selected depending on the type and size of the biomolecule to be fused.

The type of the antigen is not particularly restricted as long as it can interact with the VL-region polypeptide and the VH-region polypeptide, and may be appropriately selected depending on the purpose as a substance to be detected. Further, the VL-region polypeptide and the VH-region polypeptide may be appropriately selected as polypeptides capable of interacting with such an antigen.

(2) Environmentally-Responsive Substance

The “environmentally-responsive substance” as used in the invention means a substance whose state changes depending on the environment around the substance. Examples of environmental changes which can be used in the present invention include changes in steric structure, denaturation, phosphorylation state and phase changes. For example, if the environmentally-responsive substance is a fluorescent dye, the fluorescence intensity and/or the fluorescence wavelength may be changed by a change in the steric structure, denaturation, phosphorylation state, a phase change thereof or the like. For detection of an antigen with high versatility, the environmentally-responsive substance preferably utilizes the binding reaction of a VH-region polypeptide and a VL-region polypeptide with the antigen, and is more preferably a substance whose state changes due to a phase change caused by the binding reaction, for example, forming a hydrophobic environment.

The environmentally-responsive substance which can be used in the present invention is preferably an environmentally-responsive luminescent substance in view of accuracy and simplicity of detection. Such a luminescent substance may be either a phosphorescent substance or a fluorescent substance, and more preferably a fluorescent substance in view of the changing ratio of the luminescence intensity upon the environmental change.

Various commercial products of the above-mentioned environmentally-responsive substance are available, and the environmentally-responsive substance may be arbitrarily selected based on the information from literatures or from the commercially-available products, depending on the sequences of the VH-region polypeptide and VL-region polypeptide to be used, the type of the antigen and the environmental change to be applied. Examples thereof include a microenvironmental probe described in “Fluorescence Measurement (Keiko Sokutei)”, 1983, Japan Scientific Societies Press.

Specific examples of the environmentally-responsive substance include fluorescein and derivatives thereof; Dapoxyl dyes and derivatives thereof; Dansyl dyes and derivatives thereof; naphthalene and derivatives thereof; fluorescamine and derivatives thereof; naphthofluorescein and derivatives thereof; aminocoumarin derivatives; hydroxycoumarin and derivatives thereof; BODIPY derivatives; benz-2-oxa-1,3-diazole and derivatives thereof; Oregon Green and derivatives thereof; pyridyloxazole and derivatives thereof; and pyrene and derivatives thereof These environmentally-responsive substances may be used singly or as a combination of two or more thereof, as long as each environmentally-responsive substance is used for only one of the VH-region and VL-region polypeptides.

Among these, the environmentally-responsive substance is preferably a hydrophobic field-responsive probe whose luminescence intensity increases due to the phase change to the hydrophobic field. Examples of the hydrophobic field-responsive probe include Dapoxyl dyes and derivatives thereof; Dansyl dyes and derivatives thereof; and fluorescein and derivatives thereof A fluorescent dye such as a Dapoxyl dye or a Dansyl dye is preferable since it emits only a low amount of luminescence before binding of the antigen in a hydrophilic environment and is sensitive to environmental changes and stable, so that the amount of luminescence before binding of the antigen (background luminescence or background noise) is about 0 and the S/N ratio (signal-to-noise ratio) upon the detection can be extremely high, and a Dapoxyl dye is more preferable since the influence of its excitation wavelength on a polypeptide is small.

Specific examples of the environmentally-responsive substance used in the invention include allyl naphthalene sulfonate such as 1-anilinonaphthalene-8-sulfonate (ANS), N-methyl-2-anilinonaphthalene-6-sulfonate (MANS) and 2-p-toluidinylnaphthalene-6-sulfonate (TNS); dimethylaminonaphthalene sulfonate; nitrobenzofurazan (NBD); Dapoxyl dyes (e.g., benzenesulfonic acid, 4-[5-[4-(dimethylamino)phenyl]-2-oxazolyl); Dapoxyl derivatives such as Dapoxyl sulfonyl chloride, Dapoxyl succinimidyl ester, Dapoxyl 3-sulfonamidopropionic acid, Dapoxyl (2-bromoacetamidoethyl) sulfonamide and Dapoxyl (2-aminoethyl) sulfonamide; Dansyl dyes such as dansyl chloride, dansyl sulfonamide, dansylaminoethyl-3-phosphate, 1-dansylsulfonamide-3-N,N-dimethylaminopropane, dansyl choline, dansyl galactoside, dansyl lysine and dansyl phosphatidyl ethanolamine; and fluorescein.

The method for labeling the VL-region polypeptide or the VH-region polypeptide with the environmentally-responsive substance is appropriately selected depending on the type of the environmentally-responsive substance to be used. When the environmentally-responsive substance is a nonpeptidic compound, the antibody molecule (i.e., the VL-region or VH-region polypeptide) can be labeled by a known method such as chemical modification of a thiol group or an amino group therein using a functional group such as maleimide or succinimide. When the environmentally-responsive substance is a peptidic compound such as a fluorescent protein, a fusion protein of the peptidic compound with the VL-region polypeptide or VH-region polypeptide may be prepared and used. The fusion protein may be prepared by any known method.

The environmentally-responsive substance may be bound to any site of the VH-region or VL-region polypeptide chain as long as the binding of the VH-region or VL-region polypeptide with the antibody is not inhibited. For example, the environmentally-responsive substance may be directly linked to the VL-region polypeptide or the VH-region polypeptide, or may be linked to the VL-region polypeptide or the VH-region polypeptide via a spacer. By using such a spacer, it is possible to appropriately adjust the position of the environmentally-responsive substance on the VH-region or VL-region polypeptide chain so that an environmental change occurs when the VL-region polypeptide and the VH-region polypeptide are bound to the antigen by an antigen-antibody reaction. Examples of such a spacer include flexible hydrophilic molecules such as polyethylene glycol derivatives and peptides, and polyethylene glycols are preferred in view of prevention of nonspecific adsorption.

Regarding the site on the VH-region or VL-region polypeptide to which the environmentally-responsive substance is bound or linked (i.e., “linking site”), in view of variability of the types of detectable antigens, one of the VH-region and VL-region polypeptides is preferably labeled such that the environmentally-responsive substance is located at the interface between the VL-region polypeptide and the VH-region polypeptide when the VL-region polypeptide and the VH-region polypeptide cooperatively form a complex with the antigen, and the site is appropriately selected depending on the purpose. The VH-region polypeptide and the VL-region polypeptide cooperatively recognize the antigen to form a complex. At this time, the complex is composed of a portion at which the antigen is bound to (recognized by) each polypeptide (that is, the “antigen recognition site” of the complex) and portions including only the polypeptides. In the invention, the term “interface” means a region on the VH-region and VL-region polypeptides other than the antigen recognition site of the complex, at which the VL-region polypeptide and the VH-region polypeptide face each other when the complex is formed.

By labeling at this interface, the environmentally-responsive substance positioned inside the complex when the complex is formed, whereby the degree of the environmental change can be increased compared to cases where each polypeptide exists independently. Further, since the influence of the solvent present around the complex can be minimized, a decrease in luminescence intensity can be minimized when a luminescent substance as mentioned below is used. As a result, detection can be performed regardless of the size of the antigen, the hydrophilicity/hydrophobicity of the antigen, and the like, whereby the antibody can be made to be applicable to a wide range of antigens.

Examples of the method for labeling with the environmentally-responsive substance at a site near such an interface between the VH-region polypeptide and the VL-region polypeptide include a method wherein a surrounding pH is adjusted when the labeling is carried out, and a method wherein modified amino acids are prepared using environmentally-responsive substances to be labeled having various modified functional groups. Among these, the method by pH adjustment is preferred since it is possible to specify the labeling site depending on the position of a polar amino acid in the polypeptide. Further, to adjust the labeling site by pH adjustment, an appropriate polar amino acid may be added to the interface or the vicinity of the interface between the VH-region polypeptide and the VL-region polypeptide. Polar amino acids are well-known in the art and may be any of, for example, a neutral amino acid such as serine, tyrosine or cysteine; a basic amino acid such as histidine and lysine; or an acidic amino acid such as aspartic acid or glutamic acid. The polar amino acid to be labeled by the environmentally-responsive substance is preferably serine, cysteine or lysine in view of simplicity of site-specific labeling.

The polypeptide labeled with the environmentally-responsive substance may be one of the VH-region polypeptide and the VL-region polypeptide, and either the VH-region polypeptide or the VL-region polypeptide may be labeled.

Examples of the method for labeling a site near the interface between the VH-region polypeptide and the VL-region polypeptide with the environmentally-responsive substance also include a position-specific labeling method as disclosed in Nature Methods Vol. 3, 923-929 (2006), which may be selected as appropriate.

More specifically, it is possible to attach a single molecule of a fluorescent dye to a specific site of the VH-region or VL-region polypeptide in a pinpoint manner by using a 4-base-codon-recognizing tRNA to which a fluorescence-labeled amino acid is linked.

Further, the site labeled with the environmentally-responsive substance in each polypeptide can be easily confirmed by, for example, using a mass spectrometer (MS) and comparing the molecular weight expected from the amino acid sequence of the biological substance with data obtained from MS, thereby specifying the amino acid labeled with the environmentally-responsive substance.

The unlabeled polypeptide and the labeled polypeptide may be brought into contact with the antigen under a condition where the polypeptides are dispersed in the sample or may be brought into contact with the antigen under a condition where each of the unlabeled and labeled polypeptide is immobilized on a solid phase. Since the unlabeled polypeptide and the labeled polypeptide in either a dispersed state or an immobilized state cooperatively recognize the antigen, the unlabeled polypeptide and the labeled polypeptide bind to the antigen with higher affinity than in cases where single chains thereof independently recognize the antigen.

The available ratio of existence between the labeled polypeptide and the unlabeled polypeptide at the time of the contact is generally 1:1. Further, in terms of the ratio of existence between the labeled polypeptide and the unlabeled polypeptide at the time of the contact, the amount of the unlabeled polypeptide may be preferably from 1 to 1000 times, more preferably from 1 to 100 times, and still more preferably from 2 to 10 times larger than that of the labeled polypeptide, in view of enhancement of the sensitivity of the labeled polypeptide to the environmental change and reduction in detection time. Addition of a large amount of the unlabeled polypeptide does not cause increase in the background of the fluorescence, while increasing the collision frequency between the unlabeled peptide and the antigen, whereby the time required for the detection is reduced.

The antigen to be detected is not restricted as long as it can be recognized by the VH-region polypeptide and the VL-region polypeptide, and may generally be any substance which can be recognized by an antibody molecule, such as a low molecular weight protein, high molecular weight protein or glycoprotein. The VH-region polypeptide and the VL-region polypeptide to be used are selected based on the antigen to be detected.

The sample used in the antigen detection method of the present invention is not restricted as long as it is a liquid sample usually used for biological molecules such as polypeptides, and examples thereof include appropriate buffers such as phosphate buffer, HEPES buffer and physiological saline. If applicable, it may be a sample derived from a living body, such as a blood sample, plasma sample, body fluid sample or the like itself or one prepared by dilution thereof with the above-mentioned buffer or the like.

The condition under which the labeled polypeptide and the unlabeled polypeptide are brought into contact with the antigen in a sample is also not restricted as long as a pair of antibody molecules corresponding to the labeled polypeptide and the unlabeled polypeptide can recognize the antigen.

In the detection step of the antigen detection method of the present invention, detection of a change in the environmentally-responsive substance due to an environmental change caused by recognition of the antigen by the unlabeled polypeptide and labeled polypeptide is carried out.

That is, when the unlabeled polypeptide and the labeled polypeptide cooperatively recognize the antibody and come into contact therewith, a complex having a unique steric structure is formed. Due to the formation of the complex, an environmental change occurs around the labeled polypeptide that is a part of the complex.

The detection of a change in the environmentally-responsive substance in the antigen detection method of the invention is not restricted as long as the detection is based on a change in the environmentally-responsive substance, and a system normally used for detection of a change in property of the environmentally-responsive substance may be applied thereto. For example, in cases where a luminescent substance is used as the environmentally-responsive substance, a method usually used for detection of emission from the luminescent substance can be applied.

Further, any detection is included in the detection step of the present invention as long as the measurement can be carried out based on the amount of change in the environmentally-responsive substance. Such detection includes not only detection of existence (presence or absence) of the antigen, but also quantification of the antigen concentration in cases where the amount of change is correlated with the amount of the antigen. Further, confirmation of changes in the antigen concentration with time by carrying out continuous detection is also included in the detection of the present invention. Such a detection system can be easily carried out by those skilled in the art by application of known technologies.

Since, in the antigen detection method of the invention, the detection is possible without using a reaction such as FRET or BRET, the other polypeptide of the pair of polypeptides does not necessarily need to be labeled with the environmentally-responsive substance. Therefore, background noise due to autofluorescence, which is problematic in the case of FRET or the like, is not generated even when a large amount of the unlabeled polypeptide is used. As a result, decrease in the antigen detection sensitivity is unlikely to occur, and the time required for the detection reaction can be reduced. Further, the measurement can be carried out with a simple measuring instrument, which leads to compaction of the instrument and inexpensive detection.

The antibody fragment polypeptide set of the present invention includes: an unlabeled polypeptide which is one of a pair of separate VH-region polypeptide and VL-region polypeptide which are capable of cooperatively recognizing an antigen; and a labeled polypeptide which is the other of the pair of separate VH-region polypeptide and VL-region polypeptide and which is labeled with an environmentally-responsive substance at a site where the environmentally-responsive substance does not inhibit binding of the antigen.

Since, the present antibody fragment polypeptide set provides the unlabeled polypeptide and the labeled polypeptide which can be used for the antigen detection method, the antigen detection method can be simply carried out using the kit.

The unlabeled polypeptide and the labeled polypeptide included in the antibody fragment polypeptide set are the unlabeled polypeptide and the labeled polypeptide used in the antigen detection method described above. The same descriptions and definitions as described above for the antigen detection method are applied to the VH-region polypeptide and the VL-region polypeptide, which are used as the labeled or unlabeled polypeptide, and the environmentally-responsive substance used in the antibody fragment polypeptide set.

Further, the antigen detection method of the invention can be preferably applied to a device having a detection mechanism for detection of a change in an environmentally-responsive substance.

That is, the antigen detection device of the invention includes: an unlabeled polypeptide which is one of a pair of separate VH-region polypeptide and VL-region polypeptide which are capable of cooperatively recognizing an antigen; a labeled polypeptide which is the other of the pair of separate VH-region polypeptide and VL-region polypeptide and which is labeled with an environmentally-responsive substance at a site where the environmentally-responsive substance does not inhibit binding of the antigen; and a detection unit that detects, when a complex is formed by contact of the unlabeled polypeptide and the labeled polypeptide with the antigen, a change in the environmentally-responsive substance caused by a change in the environment around the labeled polypeptide after the contact.

Since the present antigen detection device has the detection unit that detects, when the complex of the unlabeled polypeptide, the labeled polypeptide and the antigen is formed, a change in the environmentally-responsive substance caused by a change in the environment around the labeled polypeptide after the contact, the antigen can be detected based on the degree of change in the environmentally-responsive substance detected by the detection unit.

One example of the antigen detection device of the present invention will now be described referring to drawings.

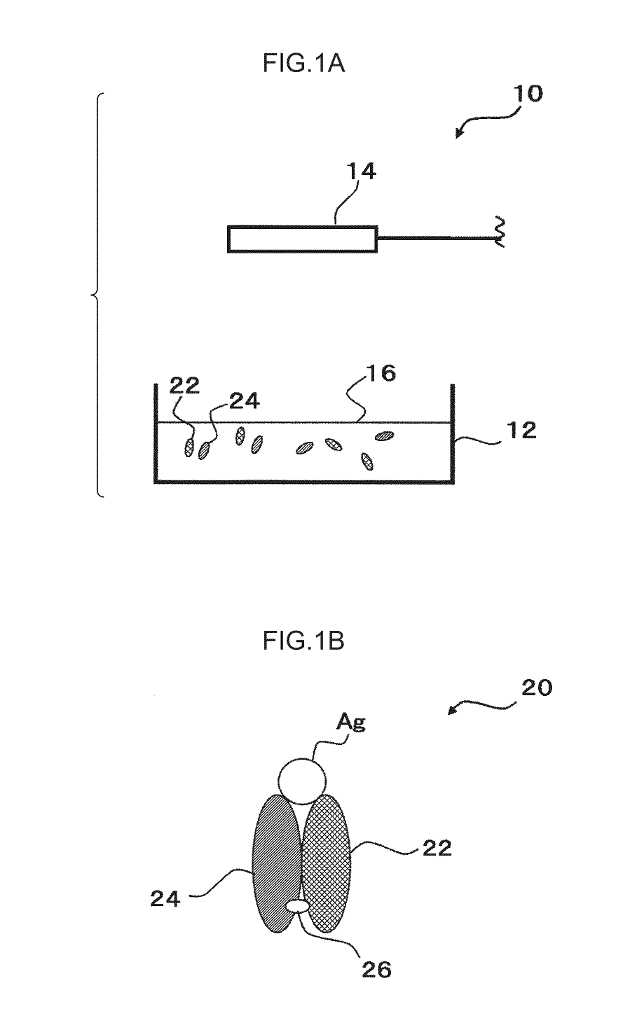

FIG. 1A shows an antigen detection device 10. The antigen detection device 10 has at least: a storage container 12 that stores a sample solution 16; and a detection unit 14 that detects a change that occurs in the storage container 12. To the detection unit 14, a control unit (not shown) that controls the entire antigen detection device 10 is connected.

The sample solution 16 stored in the storage container 12 contains at least unlabeled polypeptides 22 and labeled polypeptides 24, and an environmentally-responsive substance 26 is linked to each of the labeled polypeptides 24 (see FIG. 1B). The unlabeled polypeptide 22 and the labeled polypeptide 24 come closer to each other when they respectively recognize an antigen Ag, and are bound to the antigen Ag to form a complex 20. The same descriptions or definitions as described above are applied as they are to the unlabeled polypeptide 22, the labeled polypeptide 24, the environmentally-responsive substance 26, the antigen Ag and the sample solution 16, respectively.

The shape or the like of the storage container 12 is not particularly restricted as long as the storage container 12 can store the sample solution 16, and may be dish-shaped or tube-shaped.

Further, the storage container 12 may be integrated in the antigen detection device 10 or may be attachable to and detachable from the body of the antigen detection device 10.

The detection unit 14 is appropriately selected depending on the type of the environmentally-responsive substance 26. For example, when the environmentally-responsive substance 26 is a luminescent substance, a detection unit 14 having a mechanism that can detect light, as the change occurring in the storage container 12, emitted from the luminescent substance is selected, and when the environmentally-responsive substance 26 is a fluorescent substance, a detection unit 14 having a mechanism that can detect fluorescence, as the change occurring in the storage container 12, emitted from the fluorescent substance is selected. Such a detection unit 14 may be a photosensor usually used for detection of luminescence or fluorescence.

When the change in the environmentally-responsive substance 26 is a chemical change, the detection unit 14 may be another sensor applicable to chemical detection, such as a pH sensor or a concentration sensor. When a sensor of which sensing is based on its contact with the sample solution 16 (for example, a pH sensor or a concentration sensor) is used, the detection unit 14 may be placed inside the storage container 12.

The detection unit 14 has a calculation mechanism (not shown), which calculation mechanism enables calculation of the degree of change of the environmentally-responsive substance 26 that occurs in the storage container 12 and output of the result of the calculation to a control unit (not shown) as the detection result. Further, the control unit (not shown) is connected to respective components such as a result display unit of the antigen detection device 10 and controls driving of the entire antigen detection device 10.

In the antigen detection device 10, when the sample solution 16 is put in the storage container 12, and an instruction to start the detection is input, detection of the antigen Ag starts. After the start of the detection, changes in the environmentally-responsive substance 26 present in the storage container 12 are detected by the detection unit 14.

When the antigen Ag exists in the sample solution 16 stored in the storage container 12, the unlabeled polypeptide 22 and the labeled polypeptide 24 respectively recognize the antigen Ag and come closer to each other, thereby forming a complex 20 together with the antigen Ag (see FIG. 1B). At this time, the environment around the environmentally-responsive substance 26 of the labeled polypeptide 24 changes. The detection unit 14 detects the change, calculates a degree of change in the environmentally-responsive substance 26 occurred in the storage container 12 and outputs the degree of change or the existence of the antigen Ag to the control unit. The degree of change or the existence of the antigen Ag is displayed on the result display unit by the control unit.

When the antigen Ag is absent in the sample solution 16 stored in the storage container 12, the molecules of unlabeled polypeptide 22 and the molecules of labeled polypeptide 24 are kept dispersed, and do not form the complex 20 even when they come closer to each other. Therefore, the environment around the environmentally-responsive substance 26 on the labeled polypeptide 24 does not change. In this case, there is no change in the environmentally-responsive substance 26 in the storage container 12; therefore, the detection unit 14 does not detect a change. Therefore, the detection unit 14 outputs the absence of detection of the degree of change or the absence of the antigen Ag to the control unit, and the absence of detection of the degree of change or the absence of the antigen Ag is displayed on the result display unit by the control unit.

Thus, in the antigen detection device 10, the existence or absence of the antigen Ag in a sample can be detected.

Although the unlabeled polypeptide 22 and the labeled polypeptide 24 are dispersed in the sample solution 16 in the antigen detection device 10, the invention is not restricted thereto. Hereinbelow, an embodiment of the antigen detection device in which the unlabeled polypeptide 22 and the labeled polypeptide 24 are individually immobilized is described with reference to an immobilization support 30 which can be placed in the storage container 12.

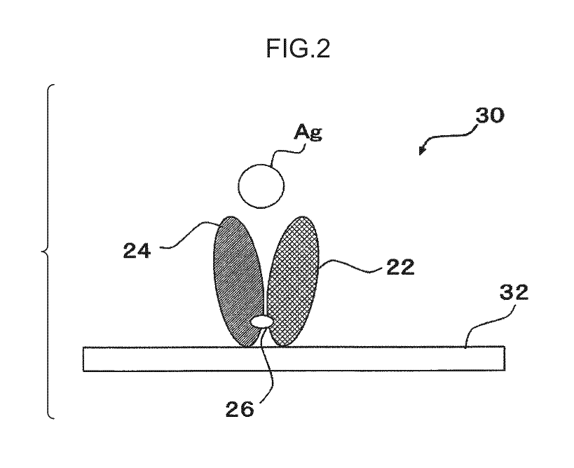

FIG. 2 shows the immobilization support 30 which can be placed in the storage container 12. The immobilization support 30 has at least: a support 32; and the unlabeled polypeptide 22 and the labeled polypeptide 24 independently immobilized on the support 32 in a positional relationship that allows each of the unlabeled polypeptide 22 and the labeled polypeptide 24 to bind to the same antigen Ag. The above descriptions and definitions of the unlabeled polypeptide 22, the labeled polypeptide 24, the environmentally-responsive substance 26, the antigen Ag and the sample solution 16 may be applied without modification to the same elements of the present embodiment.

The support 32 is not particularly restricted as long as the labeled polypeptide 24 and the unlabeled polypeptide 22 can be immobilized thereon in a prescribed relative position, and known supports usually used for immobilization of various polypeptides may be applied thereto. Examples of materials of the support 32 include glass, metal oxides such as silica, alumina, titania, zirconia and indium tin oxide (ITO); metal nitrides such as silicon nitrides, gallium nitrides, aluminum nitrides and indium nitrides; and porous materials such as ceramics and polysulfones. Further, the support 32 may be a support formed from any of the metal oxides and metal nitrides on which a known self-assembled monolayer formed using an alkanethiol or the like, or may be that further having a hydrophilic polymer such as a polysaccharide (e.g., carboxymethyl cellulose) formed on the self-assembled monolayer.

In the immobilization support 30, the unlabeled polypeptide 22 and the labeled polypeptide 24 are independently immobilized on the support 32 via binding points. The binding points are sites of the polypeptides other than the sites which recognize the antigen Ag (i.e., antigen recognition sites), and the binding points have a positional relationship which allows the polypeptides to cooperatively bind to the antigen Ag. As a result, each of the unlabeled polypeptide 22 and the labeled polypeptide 24 immobilized on the support 32 via the binding points can move independently, while they can come closer to each other upon recognition of the antigen Ag and cooperatively bind to the antigen Ag.

Thus, in the immobilization support 30, the antigen Ag can be detected as in cases where the unlabeled polypeptide 22 and labeled polypeptide 24 dispersed in the sample solution are used. Further, since the unlabeled polypeptide 22 and the labeled polypeptide 24 are immobilized on the support, a washing operation can be carried out after binding of the antigen Ag, and measurement can be repeatedly carried out, as compared to the cases where the polypeptides dispersed in the sample solution are used. Further, the unlabeled polypeptide 22 and the labeled polypeptide 24 are each immobilized on the support such that they can cooperatively bind to the antigen Ag and move independently; therefore, when the antigen Ag comes closer and capable of being bound to the polypeptides, the unlabeled and labeled polypeptides can cooperatively bind to the antigen Ag with a higher affinity than in cases where a polypeptide solely binds to the antigen Ag, and the antigen Ag can be detected.

In the above, the immobilization support 30 is described as being in a form to be placed in the storage container 12. However, the support's form is not restricted thereto, and the support may constitute a part of the storage container 12, or the storage container 12 may be provided in a part of the immobilization support 30.

The production process of the immobilization support 30 is described hereinbelow, omitting the symbols.

The immobilization support is preferably produced by a method including:

bringing an unlabeled polypeptide and a labeled polypeptide into contact with an antigen to form a complex of the unlabeled and labeled polypeptides bound to the antigen, wherein the unlabeled polypeptide being one of a pair of separate VH-region and VL-region polypeptides capable of recognizing the one type of antigen, and the labeled polypeptide being the other of the pair of VH-region polypeptide and VL-region polypeptide and being labeled with an environmentally-responsive substance at a site where the environmentally-responsive substance does not inhibit binding of the antigen (formation step);

immobilizing the complex on a support via the polypeptides in the complex (immobilization step); and

removing the antigen from the complex to obtain an immobilization support on which the polypeptides are independently immobilized in the same positional relationship as that in the case where the polypeptides are bound to the antigen (removal step).

By this production process, the antigen is removed after immobilization of the complex including the separate polypeptides and the antigen on the support; therefore, the immobilization support of the present invention on which the polypeptides are each immobilized in a positional relationship which allows their binding to the antigen can be easily prepared.

In the formation step in the production process of the immobilization support wherein the complex is formed, the complex including the polypeptides and the antigen may be formed by a known method, specifically, by mixing the unlabeled and labeled polypeptides with the antigen.

The mixing ratio between the VH-region polypeptide or VL-region polypeptide and the antigen may be appropriately set depending on the form of binding to the antigen. The ratio of the number of antigens to the valency of a molecule formed by a combination of the VH-region and VL-region polypeptides is from 0.1:1 to 10:1, preferably from 0.1:1 to 1:1, and more preferably from 0.1:1 to 0.3:1, in view of efficiency and detection sensitivity. On the other hand, for an antigen which has a low affinity in general or is expected to be difficult to directly immobilize to the support, such as when using a low molecular weight compound as the antigen, a larger amount of the antigen is preferably used, and the ratio of the number of antigens to the valency is preferably from 0.5:1 to 5:1.

Here, “the valency of a molecule formed by a combination of the VH-region and VL-region polypeptides” means the number of the antigen-binding sites present in one polypeptide or in the molecule formed by a combination of polypeptides. That is, when a molecule formed by a combination of polypeptides constitutes a complete antibody molecule, the valency of the molecule is equal to that of the antibody molecule. When one polypeptide or a molecule formed by a combination of polypeptides does not constitute a complete antibody molecule, the valency of the one polypeptide or molecule is considered to be 1 as long as one antigen-binding site is included therein.

The complex may be formed by any number of molecules; however, in order to facilitate the control of the quantitative ratio, the number of molecules forming the complex is preferably three molecules such as two types of the polypeptides and one antigen, but not limited thereto.

For example, when an anti-lysozyme VH-region polypeptide, an anti-lysozyme VL-region polypeptide and lysozyme are used, the VH-region polypeptide and the VL-region polypeptide interact with the antigen in a relationship (VH-region polypeptide:VL-region polypeptide) of 1:1, such that the VH-region polypeptide and the VL-region polypeptide have a valency of 1 in combination. Therefore, by mixing the VH-region polypeptide, the VL-region polypeptide and the antigen together in equal amounts in terms of number ratio, that is, at a ratio (VH-region polypeptide:VL-region polypeptide:antigen) of 1:1:1 in an aqueous solution, the complex can be easily obtained. To prevent direct immobilization of a lysozyme on the support, which leads to a decrease in the binding rate with respect to the antibody fragment, the number of the antigen is preferably lower than the valency of the antibody fragment. The ratio of the number of the antigen to the valency of the antibody molecule, which includes the VH-region and VL-region polypeptides, is more preferably from 0.1:1 to 0.9:1, and still more preferably from 0.1:1 to 0.3:1.

When immobilizing the complex, the complex formed by the procedure described above is linked to the support by a reaction appropriately selected in accordance with the type of functional group provided to the support. Since the antigen-recognition site of the polypeptide is protected by the antigen bound thereto, no additional protective treatment is required.

The method for binding the complex to the support can be selected depending on the type of the support, and is obvious to those skilled in the art. Examples thereof include, but are not restricted to, a method wherein a carboxyl group is activated by 1-(3-dimethylaminopropyl)-3-ethylcarbodiimide (EDC) or the like to bind it to an amino group, and a method wherein the binding is carried out by the reaction between a maleimide group and a thiol group.

When removing the antigen from the complex, the complex is immobilized on the support and then the antigen is removed. Since each of the antibody fragments is independently immobilized on the support, the antigen can be readily removed. Therefore, when the support is used as the immobilization support, binding reproducibility of the antigen may not decrease.

The removal of the antigen is readily carried out by using an appropriate washing solution. Any solution can be used as the washing solution as long as it reduces the avidity of the antigen and the antibodies in the complex. Examples of a condition for reducing the avidity include altering a pH toward an acidic side or an alkali side, and/or increasing a salt concentration. The washing solution varies depending on the type of the polypeptide and antigen or the like, and examples thereof include an acidic glycine buffer with which the pH may be adjusted to 2 or less; an alkaline NaOH solution with which the pH may be adjusted to 10 or more; and a borate buffer with which the salt concentration may be adjusted to 0.5 M or more.

In addition, an acidic buffer containing arginine, a buffer containing guanidine or a buffer containing urea can be appropriately used.

Here, the condition of the washing treatment with the washing solution can be appropriately adjusted. In order not to impair the stability of the polypeptides, the time for the washing treatment is in general 10 minutes or less, and preferably one minute or less. From the viewpoint of reproducibility, the time for the washing treatment is preferably 5 seconds or more.

By using the method for preparation of the immobilization support, an immobilization support on which the polypeptides are independently immobilized in a positional relationship which allows their binding to the antigen can be easily obtained, and an immobilization support having a high affinity to the antigen can be easily obtained.

Thus, by performing an operation including immobilizing a complex formed from the antigen, the VH-region polypeptide and the VL-region polypeptide on a support via the polypeptides and removing the antigen, an immobilization support which may be used for an immunoassay employing the antigen-antibody reaction can be obtained.

The antigen detection device described in the present specification may be applied to a biosensor utilizing the binding reactivity between a polypeptide and an antigen (e.g., “Biochip and Biosensor”, 2006, Kyoritsu Shuppan Co., Ltd.). The biosensor is defined in the widest sense and means a sensor which converts an interaction between biological molecules into a signal such as an electric signal, to measure and/or detect a substance of interest. Respective applications are described hereinbelow.

A conventional biosensor is constituted by a receptor portion that recognizes a chemical substance to be detected (e.g., antigen, in the present invention) and a transducer portion that converts a physical or chemical change (e.g., change in the environmentally-responsive substance, in the present invention) occurred in the receptor portion into an electric signal. Examples of a combination of substances in a living body having affinities to each other include enzyme and substrate; enzyme and coenzyme; antigen and antibody; and hormone and receptor. In general, a biosensor utilizes the principle that one of these substances having affinities to each other is immobilized on a support and used as a molecule-recognizing substance to selectively detect the other substance as its counterpart. By applying the antigen detection device to a support being formed from, for example, a porous material such as a ceramic or polysulfone, a glass film, or a metal film and having a polypeptide immobilized on the surface thereof, the detection can be performed more easily than in the case of a conventional biosensor.

The antigen detection device can be applied to another sensor other than the above-mentioned biosensor as long as the sensor is a detection system to which the antigen detection method of the present invention can be applied.

The present invention further provides an antigen detection kit for detecting a specific antigen of interest. This antigen detection kit includes an antibody-fragment-polypeptide set including the above-mentioned labeled polypeptide and unlabeled polypeptide which have binding capacities to the antigen of interest. The antigen of interest can be easily detected using the antigen detection kit. This antigen detection kit may include separately wrapped packages containing the labeled polypeptide and the unlabeled polypeptide, respectively, or may include an immobilization support on which the labeled polypeptide and unlabeled polypeptide are immobilized.

Alternatively, the antigen detection kit may include: a VH-region polypeptide; a VL-region polypeptide; and an environmentally-responsive substance; and, optionally, a labeling agent. In the case of such an antigen detection kit, the user can select which polypeptide out of the VH-region polypeptide and the VL-region polypeptide should be used as the labeled polypeptide.

As described above, in the present invention, a labeled polypeptide labeled using an environmentally-responsive substance and an unlabeled polypeptide are used for detection of the presence/absence of an antigen; therefore, the presence/absence of the antigen can be simply judged without using various detection systems when, for example, a substance of which change due to an environmental change can be visually observed is used as the environmentally-responsive substance. Thus, the antibody-fragment-polypeptide set including the labeled polypeptide and the unlabeled polypeptide may be used to constitute the antigen detection device without any modification.

That is, according to another embodiment of the invention, the antigen detection device includes:

a pair of separate VH-region polypeptide and VL-region polypeptide capable of cooperatively recognizing one type of antigen, one of the VH-region and VL-region polypeptide being an unlabeled polypeptide and the other of which being a labeled polypeptide; and

an environmentally-responsive substance which is positioned at a site in the labeled polypeptide where the environmentally-responsive substance does not inhibit binding of the antigen, and which changes owing to an environmental change around the labeled polypeptide when the labeled polypeptide and the unlabeled polypeptide come in contact with the antigen to form a complex. In this manner, the antigen can be detected as in the above-mentioned antigen detection device, and the detection of the antigen can be carried out with a simpler constitution.

The environmentally-responsive substance used in the antigen detection device is preferably a substance which undergoes, owing to an environmental change, a change in luminescence or an emission wavelength within the visible wavelength region, or a change in temperature. Further, in the invention, an auxiliary agent and/or auxiliary component for visualization of such a change may be used. Examples of such an auxiliary agent include pH indicators and temperature-sensitive dyes.

Exemplary embodiments of the invention are described below.
<1> A method for detecting an antigen in a sample, the method comprising:

bringing an unlabeled polypeptide and a labeled polypeptide into contact with an antigen in a sample, the unlabeled polypeptide being one of a pair comprising a VH-region polypeptide and a VL-region polypeptide which are separate and capable of cooperatively recognizing the antigen, and the labeled polypeptide being the other of the pair comprising the separate VH-region polypeptide and VL-region polypeptide and being labeled with an environmentally-responsive substance at a site where the environmentally-responsive substance does not inhibit binding of the antigen, and

detecting a change in the environmentally-responsive substance caused by a change in the environment around the labeled polypeptide after the contact.
<2> The method for detecting an antigen according to <1>, wherein the change in the environment comprises formation of a hydrophobic environment, and the environmentally-responsive substance comprises a hydrophobic field-responsive probe.
<3> The method for detecting an antigen according to <1> or <2>, wherein the environmentally-responsive substance comprises a luminescent substance.
<4> The method for detecting an antigen according to <1> or <2>, wherein the environmentally-responsive substance comprises a fluorescent substance.
<5> The method for detecting an antigen according to <1> or <2>, wherein the environmentally-responsive substance comprises at least one selected from the group consisting of a Dansyl dye, a derivative of a Dansyl dye, a Dapoxyl dye, and a derivative of a Dapoxyl dye.
<6> An antibody fragment polypeptide set comprising:

an unlabeled polypeptide which is one of a pair comprising a VH-region polypeptide and a VL-region polypeptide which are separate and capable of cooperatively recognizing an antigen, and

a labeled polypeptide which is the other of the pair comprising the VH-region polypeptide and the VL-region polypeptide and which is labeled with an environmentally-responsive substance at a site where the environmentally-responsive substance does not inhibit binding of the antigen.
<7> The antibody fragment polypeptide set according to <6>, wherein the site where the environmentally-responsive substance does not inhibit binding of the antigen is located inside a complex formed by the VH-region polypeptide and the VL-region polypeptide when the antigen is cooperatively recognized by the VH-region polypeptide and the VL-region polypeptide, and is located at position on the labeled polypeptide where the VH-region polypeptide and VL-region polypeptide face each other.
<8> The antibody fragment polypeptide set according to <6> or <7>, wherein the environmentally-responsive substance comprises a hydrophobic field-responsive probe.
<9> A kit for detecting an antigen, comprising the antibody fragment polypeptide set according to any one of <6> to <8>.
<10> An antigen detection device comprising:

the antibody fragment polypeptide set according to any one of <6> to <8>, and a detection unit that, in a case in which a complex is formed by contact of the unlabeled polypeptide and the labeled polypeptide with the antigen, detects a change in the environmentally-responsive substance caused by a change in the environment around the labeled polypeptide after the contact.
<11> The antigen detection device according to <10>, further comprising a storage unit that stores a liquid comprising the unlabeled polypeptide and the labeled polypeptide.
<12> The antigen detection device according to <10> or <11>, further comprising an immobilization support which comprises a support on which the unlabeled polypeptide and the labeled polypeptide are independently immobilized in a positional relationship which allows binding of the unlabeled polypeptide and the labeled polypeptide to the antigen.
<13> The antigen detection device according to any one of <10> to <12>, wherein the environmentally-responsive substance is a luminescent substance, and the detection unit detects light emitted from the luminescent substance as a change in the environmentally-responsive substance.
<14> The antigen detection device according to any one of <10> to <13>, wherein the environmentally-responsive substance is a fluorescent substance, and the detection unit detects fluorescence emitted from the fluorescent substance as a change in the environmentally-responsive substance.
<15> The antigen detection device according to any one of <10> to <14>, wherein the environmentally-responsive substance is at least one selected from the group consisting of a Dansyl dye, a derivative of a Dansyl dye, a Dapoxyl dye, and a derivative of a Dapoxyl dye.
<16> An immobilization support, comprising:

a support; and

the antibody fragment polypeptide set according to any one of <6> to <8>,

wherein the unlabeled polypeptide and the labeled polypeptide are independently immobilized on the support in a positional relationship which allows binding of the unlabeled polypeptide and the labeled polypeptide to the antigen.

### Examples

Examples of the antigen detection method of the present invention are described hereinbelow. Unless otherwise specified, “parts” and “%” means “parts by mass” and “% by mass”, respectively.

### Example 1

(1) Preparation of Anti-Lysozyme VH-Region Polypeptide and Anti-Lysozyme VL-Region Polypeptide

Abbreviations used in the Examples are as follow:
LB: culture medium containing 1% BACTO (Registered tradename) Tryptone, 0.5% Yeast Extract and 0.5% NaCl
LBA: LB containing 100 μg/ml of ampicillin
LBAG: LB containing 100 μg/ml of ampicillin and 0.1% glucose
LBAG plate: LB agar medium containing 100 μg/ml of ampicillin and 0.1% glucose
SOC: culture medium containing 2% BACTO (Registered tradename) Tryptone, 0.5% Yeast Extract, 0.05% NaCl, 2.5 mM KCl, 20 mM glucose and 10 mM MgCl₂
PBS: 10 mM phosphate buffer (pH 7.2) containing 137 mM NaCl and 2.7 mM KCl
5% IBPBS: PBS containing 5% (v/v) IMMUNOBLOCK (trade name, manufactured by Dainippon Sumitomo Pharma Co., Ltd., Osaka, Japan)
20% IBPBS: PBS containing 20% (v/v) IMMUNOBLOCK
PBST: PBS containing 0.1% of Triton-X 100
TAE buffer: 40 mM Tris-acetate buffer (pH 8.3) containing 1 mM EDTA
TALON Buffer: 50 mM sodium phosphate buffer (pH 7.0) containing 300 mM NaCl
TALON elution buffer: TALON Buffer (pH 7.0) containing 500 mM imidazole
IPTG: isopropyl-β-thiogalactopyranoside
HBS-N buffer: 10 mM HEPES, 150 mM NaCl, pH 7.4

In all experiments, water purified with MILLI-Q (trade name, manufactured by Millipore Co., Billerica, Mass., USA) was used. Hereinafter, this purified water is referred to as milliQ water. Unless otherwise specified, general reagents used were obtained from Sigma-Aldrich Co. (St. Louis, Mo., USA), Nacalai Tesque (Kyoto, Japan), Wako Pure Chemical Industries, Ltd. (Osaka, Japan), or Kanto Chemical Co. Inc. (Tokyo, Japan). Oligo DNAs were synthesized by Texas Genomics Japan (Tokyo, Japan) or Invitrogen (Tokyo, Japan).

The genotypes of E. coli XL10-Gold and OverExpress C41 are shown in Table 1, and the primer sequences used for PCR are shown in Table 2.

TABLE 1
<E. coli.>

XL10-Gold:
Tetr Δ(mcrA) 183 Δ(mcrCB-hsdSMR-mrr) 173 endA1 supE44 thi-1 recA1
gyrA96 relA1 lac Hte [F′proAB lacIqZΔM15 Tn10 (Tet^{r}) Amy Cam^{r}]
OverExpress C41(DE3):
F⁻, ompT, hsdS_{B}(r_{B}⁻ m_{B}⁻), gal(λ cI 857, ind1, Sam7, nin5, lacUV5-T7gene1),
dcm(DE3)

TABLE 2
<Primer>

(1)
SEQ ID NO: 1
AAAAAAAGCGGCCGCGGAGCATCATCACCATCACCACCACCACCACCACT
GAGATCCGG
(The underline indicates the NotI site and the
double-underline indicates the nucleic acid
sequence corresponding to His-tag containing ten
histidine resides (His 10).)
(2)
SEQ ID NO: 2
CCAATGCTTAATCAGTGA
(3)
SEQ ID NO: 3
CTTTCTATGCGGCCCAGCCGGCCATGGCCGAKGTSVAGCTTCAGGAGTC
(The underline indicates the SfiI site.)
(4)
SEQ ID NO: 4
AAAAAAGCGGCCGCGCTCGAGACGGTGACCGTGG
(The underline indicates the NotI site.)
(5)
SEQ ID NO: 5
AAAAAAGGCCCAGCCGGCCATGGCGTCGACGGATATTTTGATGAC
(The underline indicates the SfiI site.)
(6)
SEQ ID NO: 6
TTTCTCGTGCGGCCGCACGTTTTATTTCCAACTTTG
(The underline indicates the NotI site.)

(A) Construction of Expression Plasmids

### (a) Vectors Used in the Experiments

pET-MBPp-His6: pET15b vector (Merck Chemicals Ltd., Darmstadt, Germany), into which the gene encoding maltose binding protein (MBP) tagged with His-Tag containing six histidine residues (His6) is inserted (SEQ ID NO: 7).
pIT2-LxE16: pIT2 vector (provided by MRC Cambridge, UK), into which the single-chain antibody (scFv) gene encoding anti-hen egg lysozyme (HEL) antibody LxE16 (isolated at Laboratory of Protein Engineering, Department of Chemistry and Biotechnology, Graduate School of Engineering, University of Tokyo) is inserted (SEQ ID NO: 8, amino acid: SEQ ID NO: 9).

(b) Outline of the Preparation of the Expression Vectors

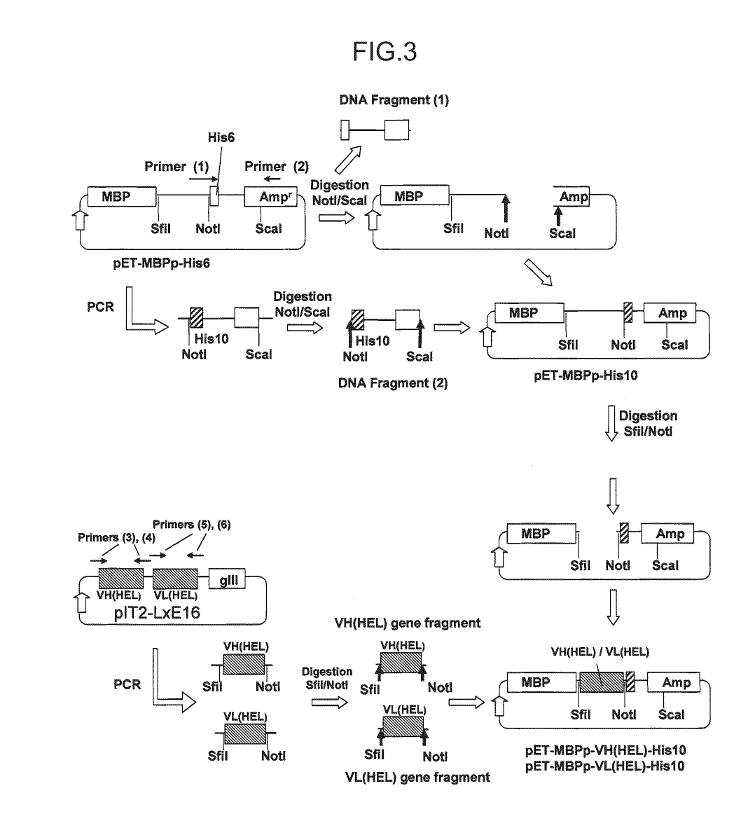

As shown in the scheme of FIG. 3, an expression vector pET-MBPp-VH(HEL)-His10 encoding a MBP-VH(HEL)-His10 protein, in which MBP and a His-tag containing ten histidine residues (His10) are respectively fused to N- and C-terminals of VH(HEL) (the heavy-chain variable region domain of the anti-lysozyme antibody LxE16), and an expression vector pET-MBPp-VL(HEL)-His10 encoding a MBP-VL(HEL)-His10 protein, in which MBP and His10 are respectively fused to N- and C-terminals of VL(HEL) (the light-chain variable region domain of the anti-lysozyme antibody LxE16) were constructed by using the pET-MBPp-His6.

First, DNA fragment (1) containing His6 was isolated from the pET-MBPp-His6, and then DNA fragment (2) encoding His10 was inserted thereinto, thereby obtaining a pET-MBPp-His10. Subsequently, a VH(HEL) gene (SEQ ID NO: 10, Table 3) was inserted into the pET-MBPp-His10, thereby obtaining the pET-MBPp-VH(HEL)-His10. Further, a VL(HEL) gene (SEQ ID NO: 11, Table 4) was inserted into the pET-MBPp-His10, thereby obtaining the pET-MBPp-VL(HEL)-His10.

TABLE 3
VH(HEL)

ATGGCCGAGGTGCAGCTTCAGGAGTCAGGACCTAGCCTCGTGAAACCTTC
TCAGACTCTGTCCCTCACCTGTTCTGTCACTGGCGACTCCATCACCAGGG
GTTACTGGAGCTGGATCCGGAAATCCCCAGGAAATAAACTTGAGTACATG
GGGTACATAAGCTACAGTGGTAGCACTTTCTACAATCCATCTCTCAAAAG
TCGAATCTCCATCACTCGAGACACATTCAAGAACCAGCTCTACCTGCAGT
TGAATTCTGTGACTACTGAGGACACAGCCACATATTATTGTGCAGAGTAC
GACGGGACTTACTGGGGCCAAGGGACCACGGTCACCGTCTC

TABLE 4
VL(HEL)

TCGACGGATATTTTGATGACCCAGACTCCAGCCACCCTGTCTGTGACTCC
AGGAGATAGCGTCAGTCTTTCCTGCAGGGCCAGCCAAAGTATTGGCAACA
ACCTACACTGGTTTCAACAAAAATCACATGAGTCTCCAAGGCTTCTCATC
AAGTATGCTTCCCAGTCCATCTCTGGGATCCCCTCCAGGTTCAGTGGCAG
TGGATCAGGGACAGATTTCACTCTCAGTATCAACACTGTGGAGACTGAAG
ATTTTGGAATGTATTTCTGTCAACAGAGTAACAGCTGGCCGTACACGTTC
GGAGGGGGGACAAAGTTGGAAATAAAACGT

(b) Isolation of DNA Fragment (1) from the pET-MBPpP-His6

To 74 μl of an aqueous solution containing about 10 μg of pET-MBPp-His6, 3 μl of ScaI (Roche Applied Science, Basel, Switzerland, 10 U), 3 μl of NotI (Roche Applied Science, 10 U), 10 μl of 10× BSA solution and 10 μl of 10× H buffer (Roche Applied Science) were added, and the mixture was then left to stand for about 3 hours at 37° C. Subsequently, the mixture was subjected to electrophoresis on 1% agarose gel (in TAE buffer), and then a DNA band of approximately 4,100 bp was excised and extracted using WIZARD SV Gel And PCR Clean-Up System (trade name, manufactured by Promega Co., Madison, Wis.). The extracted DNA was dissolved in 50 μl of milliQ water.

(c) Preparation of DNA Fragment (2)

PCR was performed by using the pET-MBPp-His6 as the template, a primer (1) (SEQ ID NO: 1) and a primer (2) (SEQ ID NO: 2). The primer (1) is a reverse primer having a nucleic acid sequence corresponding to ten histidine residues and having the NotI site; and an annealing site of the primer (1) is located downstream of the His6 coding region. The primer (2) is a forward primer; and an annealing site of the primer (2) is located approximately 500 bases downstream of the ScaI site of the pET vector.

The PCR conditions were as follows:

| | | |
|---|---|---|
| Composition of the reaction mixture | | |
| pET-MBPp-His6 (about 100 μg/ml) | 0.5 | μl |
| Primer (1) (50 μM) | 0.5 | μl |
| Primer (2) (50 μM) | 0.5 | μl |
| 10 × Pfu buffer (20 mM Mg²⁺) | 5 | μl |
| (Agilent Technologies, Inc., Santa Clara, CA) | | |
| dNTP Mixture (2.5 mM each) | 4 | μl |
| 2.5 U/μl of Pfu DNA polymerase | 0.5 | μl |
| (Agilent Technologies, Inc.) | | |
| milliQ water | 39 | μl |
| Reaction cycle | | |
| 1. 94° C., 1 min | | |
| 2. 94° C., 30 sec | | |
| 3. 58° C., 30 sec | | |
| 4. 72° C., 30 sec | | |
| (25 cycles of steps 2 to 4) | | |
| 5. 72° C., 10 min | | |
| 6. 16° C. ∞ | | |

The PCR product was purified with WIZARD SV Gel And PCR Clean-Up System and dissolved in 50 μl of milliQ water. To the solution, 1 μl of ScaI (Roche Applied Science, 10 U), 1 μl of NotI (Roche Applied Science, 10 U), 7 μl of 10× BSA solution, 7 μl of 10× H buffer (Roche Applied Science) and 4 μl of milliQ water were added, and the mixture was then left to stand for about 3 hours at 37° C. Subsequently, the mixture was subjected to electrophoresis on 1% agarose gel (in TAE buffer), and then a DNA band of approximately 1,080 bp was excised and extracted using WIZARD SV Gel And PCR Clean-Up System (Promega Co., Madison, Wis.). The extracted DNA was dissolved in 50 μl of milliQ water, thereby obtaining a solution of DNA fragment (2).

(d) Preparation of pET-MBPp-His10

0.5 μl of a solution containing the pET-MBPp-His6 from which DNA fragment (1) has been removed and 5 μl of the solution of DNA fragment (2) were mixed. Subsequently, 5.5 μl of DNA LIGATION HIGH Ver2 Solution (trade name, manufactured by TOYOBO CO., LTD., Osaka, Japan) was added to the mixture, and then DNA ligation was performed for 30 minutes at 16° C. Thereafter, about 50 μl of E. coli XL10-Gold chemical competent cells were transformed with about 1 μl of the reaction mixture. The transformants were cultured on LBAG agar medium overnight at 37° C. A single-colony transformant was further cultured in 50 ml of LBAG overnight, and then the plasmid DNA was extracted using WIZARD PLUS MINIPREPS DNA Purification Kit (trade name, manufactured by Promega Co.), thereby obtaining pET-MBPp-His10. The DNA sequence encoding His10 was confirmed in accordance with a protocol from Beckman Coulter, Inc.

(e) Restriction Enzyme Treatment of pET-MBPp-His10

To 46 μl of an aqueous solution containing about 7 μg of pET-MBPp-His110, 2 μl of SfiI (Roche Applied Science, 10 U), 6 μl of 10× BSA solution and 6 μl 10× M buffer (Roche Applied Science) were added, and the mixture was then left to stand for about 3 hours at 50° C. DNA was purified using WIZARD SV Gel And PCR Clean-Up System and then dissolved in 50 μl of an aqueous solution. To the DNA solution, 2 μl of NotI (Roche Applied Science, 10 U), 7 μl of 10× BSA solution, 7 μl of 10× H buffer (Roche Applied Science) and 4 μl of milliQ water were added, and the mixture was then left to stand for about 3 hours at 37° C. Subsequently, the mixture was subjected to electrophoresis on 1% agarose gel (in TAE buffer), and then a DNA band of approximately 4,800 bp was excised and extracted using WIZARD SV Gel And PCR Clean-Up System. The extracted DNA was dissolved in 50 μl of milliQ water.

(f) Preparation of the VH(HEL) Gene Fragment

PCR amplification of the VH(HEL) gene fragment was performed by using pIT2-LxE16 as a template, and a primer (3) and a primer (4). The primer (3) is a reverse primer having a SfiI site; and an annealing site of the primer (3) is located at the 5′ side of the VH(HEL) gene fragment. The primer (4) is a forward primer having the NotI site; and an annealing site of the primer (4) is located at the 3′ side of the VH(HEL) gene fragment.

The PCR conditions were as follows:

| | | |
|---|---|---|
| Reaction mixture composition | | |
| pIT2-LxE16 (about 100 μg/ml) | 0.5 | μl |
| Primer (3) (50 μM) | 0.5 | μl |
| Primer (4) (50 μM) | 0.5 | μl |
| 10 × Pfu buffer (20 mM Mg²⁺) | 5 | μl |
| (Agilent Technologies, Inc.) | | |
| dNTP Mixture (2.5 mM each) | 4 | μl |
| 2.5 U/μl of Pfu DNA polymerase | 0.5 | μl |
| (Agilent Technologies, Inc.) | | |
| milliQ water | 39 | μl |
| Reaction cycle | | |
| 1. 94° C., 1 min | | |
| 2. 94° C., 30 sec | | |
| 3. 58° C., 30 sec | | |
| 4. 72° C., 30 sec | | |
| (25 cycles of steps 2 to 4) | | |
| 5. 72° C., 10 min | | |
| 6. 16° C. ∞ | | |

The PCR product was purified with WIZARD SV Gel And PCR Clean-Up System and dissolved in 50 μl of milliQ water. To the solution, 2 μl of SfiI (Roche Applied Science, 10 U/μl), 7 μl of 10× BSA solution, 7 μl of 10× M buffer (Roche Applied Science) and 4 μl of milliQ water were added, and the mixture was then left to stand for about 3 hours at 50° C. The resulting DNA was purified with WIZARD SV Gel And PCR Clean-Up System and dissolved in 50 μl of aqueous solution. To the DNA solution, 2 μl of NotI (Roche Applied Science, 10 U), 7 μl of 10× BSA solution, 7 μl of 10× H buffer (Roche Applied Science) and 4 μl of milliQ water were added, and the mixture was then left to stand for about 3 hours at 37° C. Subsequently, the resulting DNA was purified with WIZARD SV Gel And PCR Clean-Up System. The extracted DNA was dissolved in 50 μl of milliQ water, thereby obtaining a solution of the VH(LxE16) gene fragment.

(g) Preparation of the VL(HEL) Gene Fragment

PCR amplification of the VL(HEL) gene fragment was performed by using pIT2-LxE16 as a template, and a primer (5) and a primer (6). The primer (5) is a reverse primer having a SfiI site; and an annealing site of the primer (5) is located at the 5′ side of the VL(HEL) gene fragment. The primer (6) is a forward primer having a NotI site; and an annealing site of the primer (6) is located at the 3′ side of the VL(HEL) gene fragment. PCR, restriction enzyme treatment and purification of DNA were performed in the same manner as in preparation of VH(LxE16) gene fragment, thereby obtaining a solution of the VL(LxE16) gene fragment.

(h) Preparation of the pET-MBPp-VH(HEL)-His10 and the pET-MBPp-VL(HEL)-His10

0.5 μl of a solution containing the restriction enzyme-treated pET-MBPp-His10 was mixed with 5 μl of a solution of VH(LxE16) or VL(LxE16). Subsequently, 5.5 μl of DNA LIGATION HIGH Ver2 Solution (TOYOBO CO.) was added to the mixture, and then DNA ligation was performed for 30 minutes at 16° C. Thereafter, about 50 μl of E. coli XL10-Gold chemical competent cells were transformed with about 1 μl of the reaction mixture. The transformants were cultured on LBAG agar medium overnight at 37° C. Single colony transformants were further cultured in 50 ml of LBAG overnight, and then the plasmid DNA was extracted using WIZARD PLUS MINIPREPS DNA Purification Kit (Promega Co.). The DNA sequences of pET-MBPp-VH(HEL)-His10 and pET-MBPp-VL(HEL)-His10 were confirmed in accordance with a protocol from Beckman Coulter, Inc.

(B) Preparation of the MBP-VH(HEL)-His10 Protein and the MBP-VL(HEL)-His10 Protein

The pET-MBPp-VH(HEL)-His10 and pET-MBPp-VL(HEL)-His10 plasmids were respectively transformed into E. coli OverExpress C41(DE3) by the heat shock method to express the genes. One μl of the plasmid (about 100 ng) and 100 μl of OverExpress C41(DE3) competent cells were mixed, and the mixture was then left to stand for 30 min on ice. Subsequently, heat shock was performed for 45 seconds at 42° C. Immediately after the heat shock, the mixture was left to stand for 2 minutes on ice. Thereafter, the cells were cured for 30 minutes by adding 200 μl of SOC medium thereto. The mixture was then spread on LBA plate and incubated overnight at 37° C.

A grown colony was inoculated into 4 ml of LBAG and cultured overnight at 30° C. with shaking 4 ml of the small-scale culture was then added to 800 ml of LBA, and cultured at large scale at 30° C. with shaking When an O.D.600 of the culture reached between 0.5 and 0.6, 400 μl of 1000 mM IPTG was added thereto and further cultured overnight at 30° C. with shaking The bacterial culture was then separated into supernatants and pellets of E. coli by centrifugation. By the methods described below, the MBP-VH(HEL)-His10 protein (SEQ ID NO: 12, Table 5) was independently collected from the supernatants by ammonium sulfate precipitation and from the pellet by ultrasonication of bacterial cells. Further, by the methods described below, the MBP-VL(HEL)-His10 protein (SEQ ID NO: 13, Table 6) was independently collected from the supernatants by ammonium sulfate precipitation and from the pellet by ultrasonication of bacterial cells.

TABLE 5
MBP-VH(HEL)-His10

MKIKTGARILALSALTTMMFSASALAKIEEGKLVIWINGDKGYNGLAEVG
KKFEKDTGIKVTVEHPDKLEEKFPQVAATGDGPDIIFWAHDRFGGYAQSG
LLAEITPDKAFQDKLYPFTWDAVRYNGKLIAYPIAVEALSLIYNKDLLPN
PPKTWEEIPALDKELKAKGKSALMFNLQEPYFTWPLIAADGGYAFKYENG
KYDIKDVGVDNAGAKAGLTFLVDLIKNKHMNADTDYSIAEAAFNKGETAM
TINGPWAWSNIDTSKVNYGVTVLPTFKGQPSKPFVGVLSAGINAASPNKE
LAKEFLENYLLTDEGLEAVNKDKPLGAVALKSYEEELAKDPRIAATMENA
QKGEIMPNIPQMSAFWYAVRTAVINAASGRQTVDEALKDAQTNSSSNNNN
NNNNNNLGPGAAHY--VEFAAQPAMADVELQESGPSLVKPSQTLSLTCSV
TGDSITRGYWSWIRKFPGNKLEYMGYISYSGSTFYNPSLKSRISITRDTF
KNQLYLQLNSVTTEDTATYYCAEYDGTYWGQGTTVTVSSAAAEHHHHHHH
HHH

### Protease Cleavage Site

TABLE 6
MBP-VL(HEL)-His10

MKIKTGARILALSALTTMMFSASALAKIEEGKLVIWINGDKGYNGLAEVG
KKFEKDTGIKVTVEHPDKLEEKFPQVAATGDGPDIIFWAHDRFGGYAQSG
LLAEITPDKAFQDKLYPFTWDAVRYNGKLIAYPIAVEALSLIYNKDLLPN
PPKTWEEIPALDKELKAKGKSALMFNLQEPYFTWPLIAADGGYAFKYENG
KYDIKDVGVDNAGAKAGLTFLVDLIKNKHMNADTDYSIAEAAFNKGETAM
TINGPWAWSNIDTSKVNYGVTVLPTFKGQPSKPFVGVLSAGINAASPNKE
LAKEFLENYLLTDEGLEAVNKDKPLGAVALKSYEEELAKDPRIAATMENA
QKGEIMPNIPQMSAFWYAVRTAVINAASGRQTVDEALKDAQTNSSSNNNN
NNNNNNLGPGAAHY--VEFAAQPAMASTDILMTQTPATLSVTPGDSVSLS
CRASQSIGNNLHWFQQKSHESPRLLIKYASQSISGIPSRFSGSGSGTDFT
LSINTVETEDFGMYFCQQSNSWPYTFGGGTKLEIKRAAAEHHHHHHHHHH

### Protease Cleavage Site

In the case of using the supernatant, 344 g of ammonium sulfate was added to about 800 ml of the culture supernatant and the mixture was stirred overnight at 4° C. Subsequently, an insoluble matter containing MBP-VH(HEL)-His10 or MBP-VL(HEL)-His10 was collected by centrifugation and the pellet was suspended in 30 ml of TALON Buffer. In the case of using the pellet of E. coli, the pellet was suspended in 30 ml of TALON Buffer and then subjected to ultrasonication, followed by centrifugation, to collect a supernatant containing MBP-VH(HEL)-His10 or MBP-VL(HEL)-His10. The supernatant was dialyzed against the TALON Buffer. Each protein collected in TALON Buffer was applied to a column (16 mm-diameter×about 15 mm-height) filled with a TALON Affinity Resin (trade name, manufactured by Clontech Laboratories, Inc., Mountain View, Calif.). Subsequently, TALON Affinity Resin, onto which the protein was adsorbed, was washed with the TALON Buffer, and then a TALON Elution Buffer was added to elute MBP-VH(HEL)-His10 or MBP-VL(HEL)-His10. The purified protein was confirmed by SDS-PAGE. The buffer of the eluate was changed to HBS-N, and then glycerol was added thereto at a final concentration of 16%. The obtained solution was stored at −80° C.

(C) Selection of Labeling Position of Environmentally-Responsive Fluorescent Dye (C-1) Alexa 647 Labeling

300 μL of the MBP-VL(HEL)-His10 solution obtained above (HBS-N, about 800 μg/ml) was labeled using Alexa Fluor (registered trademark) 647 (manufactured by Molecular Probes), by mixing them in an aqueous buffer solution. Three types of pH conditions, that is, pH 7.0, pH 8.0 and pH 10.0 were used for the labeling.

The Alexa Fluor 647-labeled MBP-VL(HEL)-His10 solution obtained above was purified with a column, and the position of Alexa Fluor 647 on VL(HEL) was identified by mass spectrometry. As a result, it was confirmed that, only under the condition of pH 7.0, binding of the antigen was not inhibited and serine in the VL region in the vicinity of the binding interface with VH was labeled, while under the conditions of pH 8.0 and pH 10.0, the vicinity of the antigen binding interface in the VL region was labeled.

(C-2) Dapoxyl Labeling

300 μL of the MBP-VL(HEL)-His10 solution obtained above (HBS-N, about 800 μg/ml) was labeled using Dapoxyl (registered trademark) (manufactured by Molecular Probes), by mixing them in an aqueous buffer solution. For the labeling, pH was set to pH 7.0 and pH 10.0.

The Dapoxyl-labeled MBP-VL(HEL)-His10 solution obtained above was purified using a column, and the position of Dapoxyl on VL(HEL) was identified by mass spectrometry. As a result, it was confirmed that, under the condition of pH 7.0, binding of the antigen was not inhibited and serine in the VL region in the vicinity of the binding interface with VH was labeled, while under the condition of pH 10.0, the vicinity of the antigen binding interface in the VL region was labeled.

(C-3) Dansyl Labeling

300 μL of the MBP-VL(HEL)-His10 solution obtained above (HBS-N, about 800 μg/ml) was labeled using Dansyl (registered trademark) (manufactured by Molecular Probes), by mixing them in an aqueous buffer solution. For the labeling, pH was set to pH 7.0.

The Dansyl-labeled MBP-VL(HEL)-His10 solution obtained above was purified using a column, and the position of Dansyl on VL(HEL) was identified by mass spectrometry. As a result, it was confirmed that, under the condition of pH 7.0, binding of the antigen was not inhibited and serine in the VL region in the vicinity of the binding interface with VH was labeled.

(D) Detection of Antigen Using Dapoxyl-Labeled MBP-VL(HEL)-His10

In the same manner as in the above (C-2), 200 μL of the MBP-VL(HEL)-His10 solution (HBS-N, about 800 μg/ml) was labeled using Dapoxyl (manufactured by Molecular Probes) at pH 7.0 at the time of labeling. Fluorescence from the obtained Dapoxyl-labeled MBP-VL(HEL)-His10 solution (HBS-N, 1.3 μM) was measured using ENVISION (manufactured by Perkin Elmer) (excitation wavelength: 405 nm, measurement wavelength: 595 nm).

Subsequently, this Dapoxyl-labeled MBP-VL(HEL)-His10 solution was mixed with the MBP-VL(HEL)-His10 solution, and a lysozyme solution was further mixed with the resulting mixture. As a result, the final concentrations of Dapoxyl-labeled MBP-VL(HEL)-His10, MBP-VL(HEL)-His10, and lysozyme in mixture were 1.3 μM, 2.6 μM and 1.3 μM, respectively. Fluorescence was measured 10 minutes after the addition of the lysozyme solution, using ENVISION (manufactured by Perkin Elmer) (excitation wavelength: 405 nm, measurement wavelength: 595 nm).

From the measured values of fluorescence before and after the addition of the MBP-VL(HEL)-His10 solution and the lysozyme solution, the rate of change in fluorescence intensity (fluorescence intensity after the addition/fluorescence intensity before the addition) was calculated. The results are shown in Table 7.

### Example 2

The rate of change in fluorescence intensity before and after the addition was calculated in the same manner as in the antigen detection by Dapoxyl-labeled MBP-VL(HEL)-His10 in Example 1 except that the final concentration of the MBP-VH(HEL)-His10 solution was 10 μM instead of 2.6 μM. The results are shown in Table 7.

### Comparative Example 1

The rate of change in fluorescence intensity before and after the addition was calculated in the same manner as in the antigen detection by Dapoxyl-labeled MBP-VL(HEL)-His10 in Example 1 except that only the lysozyme solution was added instead of the MBP-VH(HEL)-His10 solution and the lysozyme solution. The results are shown in Table 7.

### Comparative Example 2

The rate of change in fluorescence intensity before and after the addition was calculated in the same manner as in Example 1 except that only the MBP-VH(HEL)-His10 solution was added instead of the MBP-VH(HEL)-His10 solution and the lysozyme solution. The results are shown in Table 7.

### Comparative Example 3

The rate of change in fluorescence intensity before and after the addition was calculated in the same manner as in Example 1 except that pH was 10.0 instead of 7.0 when the Dapoxyl labeling was carried out. The results are shown in Table 7.

TABLE 7

| | Rate of change | |
|---|---|---|
| | in fluorescence | |
| | intensity | Note |
| Example 1 | 22% | Labeled VL, VH, antigen |
| Example 2 | 23% | Labeled VL, VH, antigen |
| Comparative Example 1 | 0% | Labeled VL, antigen |
| Comparative Example 2 | 2% | Labeled VL, VH |
| Comparative Example 3 | 0% | Labeled VL (pH 10.0), |
| | | VH, antigen |

As is evident from Table 7, in a system wherein the three factors, that is, the polypeptides and the antigen were added (Example 1), the rate of change in fluorescence intensity changed greatly, while in systems in which one or more of the three factors was not added (Comparative Example 1 and 2), the rate of change in fluorescence intensity hardly changed. Thus, according to the present invention, the rate of change in fluorescence intensity change greatly only in cases in which the three factors are added and a complex between the polypeptides and the antigen is formed, thereby allowing detection of the antigen.

Further, it can be seen from Example 2 that even in cases in which a large amount of an unlabeled polypeptide was added, the detection sensitivity did not decrease. Rather, when a large amount of an unlabeled polypeptide was added, the change in fluorescence intensity converged sufficiently within 10 minutes after the mixing. In this respect, it can be seen that the method of the present invention is advantageous compared to FRET which is known to show a decreased detection sensitivity when a large amount of a polypeptide is added due to a biased polypeptide ratio and a low average distance between polypeptide molecules.

Further, by comparison of the present Example 1 and Example 2 with Comparative Example 3, it can be seen that a high detection sensitivity can be realized by adjustment of the site to be labeled by a method such as adjustment of pH, and such an effect cannot be obtained under a common labeling condition (alkaline conditions at a pH of about 10, Comparative Example 3).

### Example 3

A Dansyl-labeled MBP-VL(HEL)-His10 solution was obtained in the same manner as in the above (C-3) except that MBP-VL(HEL)-His10 was labeled with Dansyl (manufactured by Molecular Probes) instead of Dapoxyl. This Dansyl-labeled MBP-VL(HEL)-His10 solution was mixed with the MBP-VL(HEL)-His10 solution, and a lysozyme solution was further mixed with the resulting mixture. As a result, the final concentrations of Dansyl-labeled MBP-VL(HEL)-His10, MBP-VL(HEL)-His10, and a lysozyme were 1.3 μM, 1.3 μM and 1.3 μM, respectively. The rate of change in fluorescence intensity before and after the addition was calculated in the same manner as in the antigen detection by Dapoxyl-labeled MBP-VL(HEL)-His10 in Example 1 except that fluorescence was measured using ENVISION (manufactured by Perkin Elmer) (excitation wavelength: 320 nm, measurement wavelength: 560 nm) 60 minutes after the addition of the lysozyme solution. The results are shown in Table 8.

### Comparative Example 4

The rate of change in fluorescence intensity before and after the addition was calculated in the same manner as in Example 3 except that the MBP-VH(HEL)-His10 solution, instead of the MBP-VH(HEL)-His10 solution and the lysozyme solution, was mixed with the Dansyl-labeled MBP-VL(HEL)-His10 solution. The results are shown in Table 8.

### Comparative Example 5

The rate of change in fluorescence intensity before and after the addition was calculated in the same manner as in Example 3 except that the lysozyme solution, instead of the MBP-VH(HEL)-His10 solution and the lysozyme solution, was mixed with the Dansyl-labeled MBP-VL(HEL)-His10 solution. The results are shown in Table 8.

TABLE 8

| | Rate of change | |
|---|---|---|
| | in fluorescence | |
| | intensity | Note |
| Example 3 | 14% | Labeled VL, VH, antigen |
| Comparative Example 4 | 0% | Labeled VL, VH |
| Comparative Example 5 | 0% | Labeled VL, antigen |

As is evident from Table 8, even in cases where a Dansyl dye was used instead of a Dapoxyl dye, a large change in fluorescence intensity occurred only when the three factors were added and a complex between the polypeptides and the antigen was formed, thereby allowing detection of the antigen.

Further, it is clear that, when equal amounts of the unlabeled polypeptide and the labeled polypeptide were used, although fluorescence intensity was still changing and had not converged 60 minutes after mixing of the antigen, sufficient detection sensitivity was obtained. From the relationship between Example 3 and Examples 1 and 2 described above, it can be seen that the time required for the detection can be adjusted by adjusting the amount of the unlabeled polypeptide, and that the time required for the detection can be shortened by increasing the amount of the unlabeled polypeptide.

Thus, according to the present invention, a wide variety of target molecules can be detected by a highly versatile, accurate and inexpensive method.

What is claimed is:

1. A method for detecting an antigen in a sample, the method comprising:
bringing an unlabeled polypeptide and a labeled polypeptide into contact with an antigen in a sample, the unlabeled polypeptide being one of a pair comprising a VH-region polypeptide and a VL-region polypeptide which are separate and capable of cooperatively recognizing the antigen, and the labeled polypeptide being the other of the pair comprising the separate VH-region polypeptide and VL-region polypeptide and being labeled with an environmentally-responsive substance at a site where the environmentally-responsive substance does not inhibit binding of the antigen, and
detecting a change in the environmentally-responsive substance caused by a change in the environment around the labeled polypeptide after the contact.

2. The method for detecting an antigen according to claim 1, wherein the change in the environment comprises formation of a hydrophobic environment, and the environmentally-responsive substance comprises a hydrophobic field-responsive probe.

3. The method for detecting an antigen according to claim 1, wherein the environmentally-responsive substance comprises a luminescent substance.

4. The method for detecting an antigen according to claim 1, wherein the environmentally-responsive substance comprises a fluorescent substance.

5. The method for detecting an antigen according to claim 1, wherein the environmentally-responsive substance comprises at least one selected from the group consisting of a Dansyl dye, a derivative of a Dansyl dye, a Dapoxyl dye, and a derivative of a Dapoxyl dye.

6. An antibody fragment polypeptide set comprising:
an unlabeled polypeptide which is one of a pair comprising a VH-region polypeptide and a VL-region polypeptide which are separate and capable of cooperatively recognizing an antigen, and
a labeled polypeptide which is the other of the pair comprising the VH-region polypeptide and the VL-region polypeptide and which is labeled with an environmentally-responsive substance at a site where the environmentally-responsive substance does not inhibit binding of the antigen.

7. The antibody fragment polypeptide set according to claim 6, wherein the site where the environmentally-responsive substance does not inhibit binding of the antigen is located inside a complex formed by the VH-region polypeptide and the VL-region polypeptide when the antigen is cooperatively recognized by the VH-region polypeptide and the VL-region polypeptide, and is located at position on the labeled polypeptide where the VH-region polypeptide and VL-region polypeptide face each other.

8. The antibody fragment polypeptide set according to claim 6, wherein the environmentally-responsive substance comprises a hydrophobic field-responsive probe.

9. A kit for detecting an antigen, comprising the antibody fragment polypeptide set according to claim 6.

10. An antigen detection device comprising:
the antibody fragment polypeptide set according to claim 6, and
a detection unit that, in a case in which a complex is formed by contact of the unlabeled polypeptide and the labeled polypeptide with the antigen, detects a change in the environmentally-responsive substance caused by a change in the environment around the labeled polypeptide after the contact.

11. The antigen detection device according to claim 10, further comprising a storage unit that stores a liquid comprising the unlabeled polypeptide and the labeled polypeptide.

12. The antigen detection device according to claim 10, further comprising an immobilization support which comprises a support on which the unlabeled polypeptide and the labeled polypeptide are independently immobilized in a positional relationship which allows binding of the unlabeled polypeptide and the labeled polypeptide to the antigen.

13. The antigen detection device according to claim 10, wherein the environmentally-responsive substance is a luminescent substance, and the detection unit detects light emitted from the luminescent substance as a change in the environmentally-responsive substance.

14. The antigen detection device according to claim 10, wherein the environmentally-responsive substance is a fluorescent substance, and the detection unit detects fluorescence emitted from the fluorescent substance as a change in the environmentally-responsive substance.

15. The antigen detection device according to claim 10, wherein the environmentally-responsive substance is at least one selected from the group consisting of a Dansyl dye, a derivative of a Dansyl dye, a Dapoxyl dye, and a derivative of a Dapoxyl dye.

16. An immobilization support, comprising:
a support; and
the antibody fragment polypeptide set according to claim 6,
wherein the unlabeled polypeptide and the labeled polypeptide are independently immobilized on the support in a positional relationship which allows binding of the unlabeled polypeptide and the labeled polypeptide to the antigen.


SEQUENCE LISTING (13 sequences)

SEQ ID NO: 1 - DNA, 59 bp, Artificial
  primer (1)
aaaaaaagcggccgcggagcatcatcaccatcaccaccaccaccaccactgagatccgg

SEQ ID NO: 2 - DNA, 18 bp, Artificial
  primer (2)
ccaatgcttaatcagtga

SEQ ID NO: 3 - DNA, 49 bp, Artificial
  primer (3)
ctttctatgcggcccagccggccatggccgakgtsvagcttcaggagtc

SEQ ID NO: 4 - DNA, 34 bp, Artificial
  primer (4)
aaaaaagcggccgcgctcgagacggtgaccgtgg

SEQ ID NO: 5 - DNA, 45 bp, Artificial
  primer (5)
aaaaaaggcccagccggccatggcgtcgacggatattttgatgac

SEQ ID NO: 6 - DNA, 36 bp, Artificial
  primer (6)
tttctcgtgcggccgcacgttttatttccaactttg

SEQ ID NO: 7 - DNA, 5217 bp, Artificial
  pET-MBP-His6
ctagaaataattttgtttaactttaagaaggagatatacatatgaaaataaaaacaggtgcacgcatcctcgcattatccgcattaacgacgatgatgttttccgcctcggctctcgccaaaatcgaagaaggtaaactggtaatctggattaacggcgataaaggctataacggtctcgctgaagtcggtaagaaattcgagaaagataccggaattaaagtcaccgttgagcatccggataaactggaagagaaattcccacaggttgcggcaactggcgatggccctgacattatcttctgggcacacgaccgctttggtggctacgctcaatctggcctgttggctgaaatcaccccggacaaagcgttccaggacaagctgtatccgtttacctgggatgccgtacgttacaacggcaagctgattgcttacccgatcgctgttgaagcgttatcgctgatttataacaaagatctgctgccgaacccgccaaaaacctgggaagagatcccggcgctggataaagaactgaaagcgaaaggtaagagcgcgctgatgttcaacctgcaagaaccgtacttcacctggccgctgattgctgctgacgggggttatgcgttcaagtatgaaaacggcaagtacgacattaaagacgtgggcgtggataacgctggcgcgaaagcgggtctgaccttcctggttgacctgattaaaaacaaacacatgaatgcagacaccgattactccatcgcagaagctgcctttaataaaggcgaaacagcgatgaccatcaacggcccgtgggcatggtccaacatcgacaccagcaaagtgaattatggtgtaacggtactgccgaccttcaagggtcaaccatccaaaccgttcgttggcgtgctgagcgcaggtattaacgccgccagtccgaacaaagagctggcaaaagagttcctcgaaaactatctgctgactgatgaaggtctggaagcggttaataaagacaaaccgctgggtgccgtagcgctgaagtcttacgaggaagagttggcgaaagatccacgtattgccgccaccatggaaaacgcccagaaaggtgaaatcatgccgaacatcccgcagatgtccgctttctggtatgccgtgcgtactgcggtgatcaacgccgccagcggtcgtcagactgtcgatgaagccctgaaagacgcgcagactaattcgagctcgaacaacaacaacaataacaataacaacaacctcgggccgggtgcggcacactacgtagaattcgcggcccagccggccatggccgactacaaagatattgtgctgacacagtctcctgcttccttagctgtatctctggggcagagggccaccatctcatgcagggccagccaaagtgtcagtacatctacctatagttatttacactggtaccaacagagaccaggacagccacccaaactcatcaagtatgtatccaacctagaatctggggtccctgccaggttcagtggcagtgggtctgggacagacttcaccctcaacatccatcctgtggaggaggaggatactgcaacatattactgtcagcacagttgggagattcctccgacgttcggtggaggcaccaagctggaaataaaacgtgcggccgcggtcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatctcgatcccgcgaaattaatacgactcactatagggagaccacaacggtttccctctag

SEQ ID NO: 8 - DNA, 840 bp, Artificial
  pIT2-LxE16
caaattctatttcaaggagacagtcataatgaaatacctattgcctacggcagccgctggattgttattactcgcggcccagccggccatggccgaggtgcagcttcaggagtcaggacctagcctcgtgaaaccttctcagactctgtccctcacctgttctgtcactggcgactccatcaccaggggttactggagctggatccggaaatccccaggaaataaacttgagtacatggggtacataagctacagtggtagcactttctacaatccatctctcaaaagtcgaatctccatcactcgagacacattcaagaaccagctctacctgcagttgaattctgtgactactgaggacacagccacatattattgtgcagagtacgacgggacttactggggccaagggaccacggtcaccgtctcgagcggtggaggcggttcaggcggaggtggcagcggcggtggcgggtcgacggatattttgatgacccagactccagccaccctgtctgtgactccaggagatagcgtcagtctttcctgcagggccagccaaagtattggcaacaacctacactggtttcaacaaaaatcacatgagtctccaaggcttctcatcaagtatgcttcccagtccatctctgggatcccctccaggttcagtggcagtggatcagggacagatttcactctcagtatcaacactgtggagactgaagattttggaatgtatttctgtcaacagagtaacagctggccgtacacgttcggaggggggacaaagttggaaataaaacgtgcggccgcacatcatcatcaccatcacggggccgc

SEQ ID NO: 9 - protein, 270 aa, Artificial
  Synthetic Construct
MKYLLPTAAAGLLLLAAQPAMAEVQLQESGPSLVKPSQTLSLTCSVTGDSITRGYWSWIRKSPGNKLEYMGYISYSGSTFYNPSLKSRISITRDTFKNQLYLQLNSVTTEDTATYYCAEYDGTYWGQGTTVTVSSGGGGSGGGGSGGGGSTDILMTQTPATLSVTPGDSVSLSCRASQSIGNNLHWFQQKSHESPRLLIKYASQSISGIPSRFSGSGSGTDFTLSINTVETEDFGMYFCQQSNSWPYTFGGGTKLEIKRAAAHHHHHHGA

SEQ ID NO: 10 - DNA, 341 bp, Artificial
  VH(HEL)
atggccgaggtgcagcttcaggagtcaggacctagcctcgtgaaaccttctcagactctgtccctcacctgttctgtcactggcgactccatcaccaggggttactggagctggatccggaaatccccaggaaataaacttgagtacatggggtacataagctacagtggtagcactttctacaatccatctctcaaaagtcgaatctccatcactcgagacacattcaagaaccagctctacctgcagttgaattctgtgactactgaggacacagccacatattattgtgcagagtacgacgggacttactggggccaagggaccacggtcaccgtctc

SEQ ID NO: 11 - DNA, 330 bp, Artificial
  VL(HEL)
tcgacggatattttgatgacccagactccagccaccctgtctgtgactccaggagatagcgtcagtctttcctgcagggccagccaaagtattggcaacaacctacactggtttcaacaaaaatcacatgagtctccaaggcttctcatcaagtatgcttcccagtccatctctgggatcccctccaggttcagtggcagtggatcagggacagatttcactctcagtatcaacactgtggagactgaagattttggaatgtatttctgtcaacagagtaacagctggccgtacacgttcggaggggggacaaagttggaaataaaacgt

SEQ ID NO: 12 - protein, 551 aa, Artificial
  MBP-VH
MKIKTGARILALSALTTMMFSASALAKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTNSSSNNNNNNNNNNLGPGAAHYVEFAAQPAMADVELQESGPSLVKPSQTLSLTCSVTGDSITRGYWSWIRKFPGNKLEYMGYISYSGSTFYNPSLKSRISITRDTFKNQLYLQLNSVTTEDTATYYCAEYDGTYWGQGTTVTVSSAAAEHHHHHHHHHH

SEQ ID NO: 13 - protein, 548 aa, Artificial
  MBP-VL
MKIKTGARILALSALTTMMFSASALAKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTNSSSNNNNNNNNNNLGPGAAHYVEFAAQPAMASTDILMTQTPATLSVTPGDSVSLSCRASQSIGNNLHWFQQKSHESPRLLIKYASQSISGIPSRFSGSGSGTDFTLSINTVETEDFGMYFCQQSNSWPYTFGGGTKLEIKRAAAEHHHHHHHHHH